(19) United States
(12) Patent Application Publication
(10) Pub. No.: US 2006/0165808 A1
(43) Pub. Date: Jul. 27, 2006

(54) Microcapsules for the delayed, controlled release of perindopril

(76) Inventors: Bruno Huet De Barochez, Ingre (FR); Patrick Wuthrich, Orleans (FR); Valerie Legrand, Yerres (FR); Catherine Castan, Orlienas (FR); Remi Meyrueix, Lyon (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(21) Appl. No.: 10/519,641

(22) PCT Filed: Jun. 24, 2003
(86) PCT No.: PCT/FR03/01931

§ 371 Date: Jul. 15, 2005

(30) Foreign Application Priority Data
Jun. 24, 2002 (FR) 02/07778

(51) Int. Cl. A61K 9/50; A61K 9/16

(52) U.S. Cl. 424/490

(57) ABSTRACT

The present invention relates to A microcapsule composition allowing the delayed and controlled release of perindopril, or of a pharmaceutically acceptable salt thereof, for administration by the oral route.

The present invention relates to microcapsules allowing the delayed and controlled release of perindopril, or of a pharmaceutically acceptable salt thereof, for administration by the oral route.

More precisely, the invention relates to a delayed and controlled release microparticulate form of perindopril or a pharmaceutically acceptable salt thereof for which the delayed and controlled release phases are controlled in a particular manner by means of a dual mechanism: “time-dependent” release, triggered at the end of a particular residence time in the stomach, and “pH-dependent” release, triggered by a change of pH when the particles enter the small intestine. The microparticles of the present invention are microcapsules having a granulometry of from 100 to 1200 microns that contain perindopril and are individually covered by at least one coating film allowing the delayed and controlled release of perindopril.

Perindopril in tert-butylamine salt form is marketed for the treatment of arterial hypertension and congestive heart failure. In particular it has an inhibitory activity in respect of certain enzymes, such as carboxypolypeptidases, encephalinases or kininase II. For example, by acting on the conversion enzyme it inhibits the conversion of the decapeptide angiotensin I to the octapeptide angiotensin II, which is responsible in some cases for arterial hypertension.

The therapeutic use of perindopril and pharmaceutically acceptable salts thereof allows the reduction or even suppression of the activity of the enzymes responsible for hypertensive disease or heart failure. The action on kininase II causes an increase in circulating bradykinin and also a reduction in arterial pressure by that route.

Currently, the tert-butylamine salt of perindopril is administered by the oral route in the form of an immediate-release tablet.

It is administered in the morning in one daily dose. For better treatment of arterial hypertension it is necessary not only to control the arterial pressure over the whole of the nychthemeron, but also to ensure that the treatment enables the prevention of the pressure increases observed especially in the morning, when the patients get up, to be prevented. Those pressure increases, called “early morning peaks”, are extremely difficult to control and are responsible for numerous cardiovascular accidents in hypertensive patients.

The perindopril tablets currently on the market allow pressure protection over the whole of the nychthemeron but do not enable the tablets to be completely protected from the pressure increase observed in hypertensive patients in the early morning. A clinical study of hypertensive patients has demonstrated that, using the current tablet, the plasma concentrations of active ingredient attained between 4 and 8 o'clock in the morning are not sufficient for that increase in pressure observed in the morning to disappear completely.

To solve this problem, it was necessary to develop a new galenic form, administrable once per day, guaranteeing the release and absorption of the active ingredient at the desired moment and allowing arterial hypertension to be controlled effectively over the whole of the day and in particular in the morning.

Observation shows that the majority of delayed-release forms are not capable with certainty of ensuring the release of active ingredient within a prescribed period.

Delayed release forms are, in fact, conventionally obtained by coating the active ingredient with a layer of enteric polymer, for example a copolymer of methacrylic acid and methacrylic acid methyl ester: EUDRAGIT®L. This type of enteric coating is known to have reduced permeability under the acid pH conditions of the stomach and is known to dissolve when the pH increases to a value close to that prevailing in the small intestine, thus releasing the active ingredient (AI). However, the interindividual variation of gastric pH conditions and the duration of gastric emptying do not allow for the release of AI after a defined period of time to be ensured with certainty.

The purely “time-dependent” delayed release systems, that is to say where release of the AI is triggered at the end of a defined residence time in the gastrointestinal tract, are not satisfactory either. In fact because of the intra- and inter-individual variation of gastric residence time, the release of perindopril may occur after it has passed its absorption window, which in the case of perindopril is located in the upper part of the gastrointestinal tract. Bioabsorption may thus be very weak or zero.

It would in that context be especially advantageous for a galenic form for the delayed and controlled release of perindopril to be available allowing release to be ensured with certainty by means of a dual triggering mechanism for the release of perindopril: “time-dependent” release, triggered at the end of a controlled time in the stomach, and “pH-dependent” release, triggered by an increase in pH when the galenic form passes into the intestine. Those two factors for triggering the release of perindopril placed in succession would confer on the galenic system substantial reliability of use. The release of perindopril would thus be guaranteed after a precontrolled latent period even if the change of pH were not to occur as a trigger.

It would also be advantageous for the delayed and controlled release form to be composed of a plurality of microcapsules having a diameter of less than 1200 microns. For such a form, the dose of AI to be administered is actually divided between a large number of microcapsules and as a result has the following intrinsic advantages:
the residence time of the microcapsules in the upper parts of the tract is capable of being prolonged, which ensures an increase in the time taken for the perindopril to pass through the absorption windows and hence maximises the bioavailability of the perindopril.
the use of a mixture of microcapsules having different delayed and controlled release profiles makes it possible to achieve release profiles having several waves of release or ensuring, by appropriate control of the various fractions, a constant level of plasma concentration of the AI.
the variation of gastric emptying is lower because, in this case, emptying occurs over a large number of particles and is statistically more reproducible.
placing tissue in contact with an elevated dose of perindopril, “dose dumping”, is avoided. Each microcapsule actually contains only a very reduced dose of perindopril. There is accordingly no risk of tissue deterioration caused by local over-concentration of perindopril.
it is possible to combine a plurality of galenic forms having different release kinetics (immediate release and/or delayed release and/or prolonged release) comprising a plurality of active ingredients co-administered with perindopril in such “multi-microcapsular” systems.
it is possible for the microcapsules to be presented in the form of a sachet, gelatin capsule or tablet.

Finally, it would likewise be desirable for the coating layer around the microcapsules to be of low thickness. Indeed a coating layer of considerable thickness would have a number of negative consequences
(a) the mass fraction of excipient in the galenic form would be too high, resulting in a medicament mass too large to be swallowed easily and consequently, ultimately, in problems of observance, putting the success of the treatment at risk;
(b) the time taken to produce the capsules would be very long, typically several hours.

This problem is all the more acute in the case of perindopril in view of its very high degree of solubility in aqueous media.

Accordingly, it would thus be of particular interest for a galenic form for the delayed and controlled release of perindopril to be available that simultaneously has the following properties:
the release of perindopril is capable of being triggered in two ways: by “time-dependent” release when the residence time of the particles in the stomach exceeds the desired latent period prior to the release of perindopril; or by “pH-dependent” release when the system passes into the intestine. When those two factors for triggering the release of perindopril are placed in succession, they guarantee release of the perindopril after a precontrolled latent period even if the change of pH has not occurred as a trigger;
it is composed of a plurality of coated perindopril microcapsules of small size
the mass fraction of coating excipients is limited.

The delayed or controlled release of active ingredients has been the subject of numerous works.

The application FR-A-00 14876 describes a medicament for the treatment of type II diabetes which comprises several thousand microcapsules of anti-hyperglycaemic (metformin), each microcapsule being composed of a core, comprising at least one anti-hyperglycaemic, and of a coating film (e.g. stearic acid and ethyl cellulose), applied to the core and allowing prolonged release of the anti-hyperglycaemic in vivo. Those microcapsules have a granulometry of from 50 to 1000 μm.

The application FR-A-00 14876 does not indicate how to achieve the delayed and controlled release of AI with “time-dependent” and “pH-dependent” triggering of the AI.

European Patent Application EP-A-0 609 961 discloses oral morphine granules allowing the controlled release of AI which is accelerated by an increase in pH.

The granules comprise:
a sugar core (Ø=100 to 1700 μm),
coated with an active layer containing a binder (PVP or hydroxypropyl methyl cellulose: HPMC),
and an outer casing based on:
   a polymer insoluble irrespective of pH (ethyl cellulose or copolymer of methacrylic ester and ammonium methacrylate: EUDRAGIT® RS or RL),
   an enteric polymer insoluble at acid pH (copolymer of methacrylic acid and methacrylic acid methyl ester: EUDRAGIT® L),
      a component partially soluble at acid pH (polyethylene glycol, PVP, HPMC, polyvinyl alcohol: PVA),
      optionally a plasticiser (diethyl phthalate),
      and optionally a filler (talc).

The mass fractions of AI are, for example,: 41%, 38.0%, 29.0%; and the mass fractions of outer casing are, e.g.: 14.1%, 21.5% and 12.3% (dry weight).

The release of AI occurs at all pH values and is increased when the pH changes from pH 1.2 to pH 7.5. It is thus a form of prolonged, and not delayed, release.

The patent specification U.S. Pat. No. 6,033,687 describes a formulation based on diltiazem composed of a mixture of two types of diltiazem-based granules (Ø=1.4 mm): short-latent-period granules and long-latent-period granules. The release profiles are measured at pH 1. Those granules comprise:
a neutral sugar core (Ø=0.5-1.5 mm),
a layer of diltiazem in combination with a binder (hydroxypropyl cellulose, carboxymethyl cellulose, ethyl cellulose, polyvinylpyrrolidone, alginate, EUDRAGIT®),
a single outer layer based on lubricant (talc), 2 copolymers of methacrylic ester and of ammonium methacrylate (EUDRAGIT® RS and EUDRAGIT® RL); a surfactant (sodium lauryl sulphate) and a plasticiser (triethyl citrate).

In the short-latent-period granules, the mass fraction of the coating is 12.3% as against 30.3% in the long-latent-period granules. This technique does not, however, allow long latent periods to be obtained for amounts of film-coating below 30%. Furthermore, given the intra- and inter-individual variation of gastric residence time, this “time-dependent” delayed release system may release the AI after it has passed its absorption window, the result of which is a substantial loss of bioavailability.

Patent Specification EP-B-0 263 083 describes a coating composition for microcapsules which allows an AI release profile which is of zero order and is reproducible to be obtained. The coating composition is composed of a mixture:
of a polymeric hardener, which ensures the mechanical behaviour of this membrane and which may be, e.g.,: ethyl cellulose or copolymer(s) of methacrylic acid (Eudragit E, LS or RS),
a lipophilic compound, e.g.: stearic acid or paraffin,
and talc.

This coating composition is present in the microcapsules in an amount of from 15 to 35% by weight (dry), for example. The polymer hardener/lipophilic compound ratios are, for example, 44 and 42%, respectively, in Examples 4 and 5.

The profiles obtained are profiles without latent periods of variable duration. There is no information or mention of how to obtain a delayed and controlled release profile triggered at the end of the latent period and/or by a change of pH.

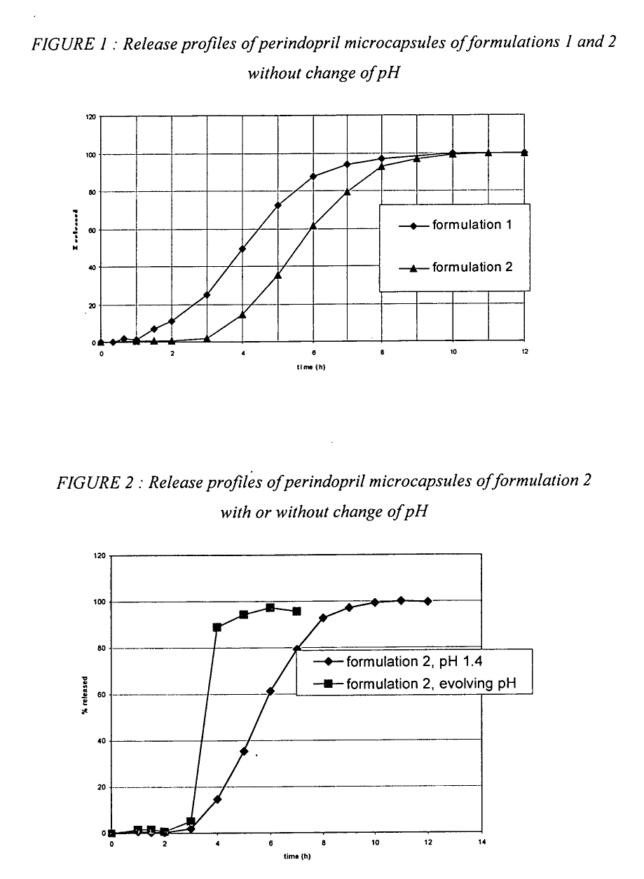

The application W0 01/58424 A1 discloses “floating” microcapsules coated with an enteric coating, for example based on Eudragit® L, magnesium stearate, talc and a plasticiser such as dibutyl sebacate. This coating may be covered in a “bioadhesive” film based on chitosan for example. Like any enteric coating, the purpose of the enteric coating according to W0 01/58424 is “pH-dependent” release and not a combination of “time-dependent” and “pH-dependent” release. Furthermore, FIGS. 1 to 3 of that application show that the simple objective of “pH-dependent” release is very imperfectly achieved, since up to 20% of the AI is released in two hours only at constant acid pH. Since the particles described in this application float in the stomach, their gastric residence time is described as increased, so that any “pH-triggered” release may even be feared to be absent. Finally, the release occurs in an uncontrolled manner as a result of parasitic losses of AI in the stomach.

In all of those proposed prior techniques, the release of AI is achieved either under the effect of the residence time in the gastrointestinal tract, or under the effect of an increase in pH which occurs during passage from the stomach into the small intestine. In the first case, it is not possible to have a latent period without AI release (release is not of a delay form) and it is to be feared that some of the AI will be released in vivo beyond its absorption window (upper parts of the gastrointestinal tract), and therefore not absorbed, when gastric emptying is too rapid. In the second case, if the galenic form sits in the stomach, it is not subject to a change of pH, and hence there is little or no release of AI. Clearly such a situation is unfavourable, since it amounts to the absorption of AI being too weak or even zero, and consequently to therapeutic ineffectiveness, which may prove serious. The prior art thus does not include a galenic system that allows delay of the release of, and that guarantees with certainty the release of, an active ingredient by a dual mechanism of “time-dependent” release and “pH-dependent” release.

On the other hand, no form of delayed and controlled release of antihypertensive of the type that inhibits angiotensin-converting enzyme currently exists.

Given such prior art, one of the essential objectives of the present invention is to provide a new multi-microparticulate galenic system for the oral administration of perindopril, that system being of the delayed and controlled release type that ensures the release of perindopril with certainty, owing to its dual mechanism of “time-dependent” and “pH-dependent” release. Those two factors triggering the release of perindopril placed in succession guarantee the release of perindopril after a precontrolled latent period, even if the change of pH has not occurred as a trigger.

An essential objective of the present invention is to propose a galenic form composed of a plurality of microcapsules that allows perindopril to be released at pH 1.4 in accordance with a delayed release profile that has a latent period of which the duration is adjustable between 1 and 8 hours, preferably from 1 to 5 hours, followed by a release phase of which the half-release time t_{1/2} is between 0.5 and 25 hours.

An essential objective of the present invention is to propose a galenic form composed of a plurality of microcapsules that allows perindopril to be released in accordance with a controlled profile when the pH has changed from 1.4 to 6.8.

Another objective of the present invention is to propose a galenic form composed of a large number of microcapsules, for example of the order of several thousand microcapsules, that multiplicity statistically ensuring good reproducibility of the transit kinetics of perindopril over the whole of the gastro-intestinal tract, so that the result is better control of the bioavailability and thus better effectiveness.

An essential objective of the present invention is to propose a galenic form of perindopril composed of a plurality of coated microcapsules that avoids the use of large quantities of coating.

An essential objective of the present invention is to propose a pharmaceutical form composed of a plurality of coated microcapsules allowing perindopril to be presented in a form that is simple to swallow: a sachet, disintegrable tablet, gelatin capsule etc.

An essential objective of the present invention is to propose a pharmaceutical form composed of a plurality of coated microcapsules allowing perindopril to be mixed with a plurality of other active ingredients.

Another objective of the present invention is to propose a pharmaceutical form composed of a plurality of coated microcapsules each containing a neutral core.

The above objectives, among others, being fixed, credit is due to the inventors for making practicable, for the purpose of ensuring certain release and good bioabsorption of perindopril, a preferably multi-microcapsular galenic system having as an essential characteristic dual triggering of the release of perindopril. This represents a major advance compared with the controlled AI release systems known hitherto in which the release of AI is triggered by a single factor: the residence time in the gastrointestinal tract for some systems, a change of pH for other systems.

More especially, the present invention relates to “reservoir” microcapsules for the delayed and controlled release of perindopril or a pharmaceutically acceptable salt thereof for oral administration, characterised in that those microcapsules are:
composed of microparticles of perindopril or a pharmaceutically acceptable salt thereof each covered by at least one coating film, that coating film being formed from a composite material comprising:
   at least one hydrophilic polymer A carrying groups ionised at neutral pH,
   at least one hydrophobic compound B, and representing a mass fraction (% weight in relation to the total mass of the microcapsules) less than or equal to 40,
and have a diameter of less than 1200 microns.

The hydrophilic polymer A carrying groups ionised at neutral pH will advantageously be selected from cellulose compounds: cellulose acetate phthalate, hydroxypropyl methyl-cellulose phthalate, hydroxypropyl cellulose acetate succinate; copolymers of methacrylic acid and of a methacrylic acid ester, copolymers of methacrylic acid and of an acrylic acid ester (Eudragit® S or L) and mixtures thereof.

Preferably, the hydrophilic polymer A is a copolymer of methacrylic acid and methyl methacrylate (Eudragit® L100/Eudragit® S100) or a copolymer of methacrylic acid and ethyl acrylate (Eudragit® L100-55).

The hydrophobic compound B will advantageously be a compound selected from vegetable waxes (Dynasan®P60, Dynasan®P 116), hydrogenated vegetable oils, hydrogenated triglycerides and mixtures thereof.

Preferably, the hydrophobic compound B is a hydrogenated vegetable oil.

More especially, the coating film for the perindopril microcapsules is formed by a mixture of hydrophilic polymer A and hydrophobic compound B in which the weight ratio B/A is between 0.2 and 4, preferably between 0.5 and 2.

The ratio will be adjusted as a function of the nature of the constituents such that:
at a constant pH of 1.4, the dissolution profile comprises a latent phase of a duration greater than or equal to half an hour—preferably between 1 and 8 hours and more especially from 1 to 5 hours,
the transition, at any instant during the latent phase, from pH 1.4 to pH 6.8, results in a perindopril release phase.

One of the defining advantages of the multi-microcapsular galenic system for the delayed and controlled release of perindopril according to the invention is to cause to come into effect in vivo two factors that trigger the release of perindopril in the gastrointestinal tract (GIT), those factors being:

the residence time in the stomach: “time-triggered” release,

the change of pH: “pH-triggered” release.

Those two factors triggering the release of perindopril are in succession, so that they confer on the galenic system substantial reliability of use. The release of perindopril is thus guaranteed after a precontrolled latent period even if the change of pH has not occurred as the trigger. The problems of interindividual variation are thus overcome. The effectiveness of the medicament comprising such a galenic system is ensured, by respecting a chronobiology that is predetermined and adapted to the therapeutic performance sought.

In addition, in the case of perindopril, for which the absorption window is limited, it is especially advantageous that the form with delayed and then controlled release is a plurality of “reservoir” microcapsules and consequently has the following intrinsic advantages:
the residence time of the microcapsules in the upper parts of the tract is capable of being prolonged, which ensures an increase in the time taken for perindopril to pass through its absorption window and thus maximises its bioavailability,
the use of a mixture of microcapsules having different delayed and controlled release profiles makes it possible to achieve release profiles having several waves of release or ensuring, by appropriate control of the different fractions, a constant level of plasma concentration of perindopril,
less sensitivity of the system to the variation of gastric emptying since the emptying, which occurs here over a large number of particles, is statistically more reproducible,
the possibility of presenting the microcapsules in the form, for example, of a sachet, gelatin capsule or tablet.

While simultaneously being economically viable and easy to ingest (optimised observance), the multi-microcapsular galenic system according to the invention allows a delayed and controlled release of perindopril in the GIT to be ensured with certainty owing to two triggers, and as a result makes it possible to disregard the inter- and intra-individual variation of in vivo pH conditions during gastric emptying.

According to an especially advantageous feature of the preferred embodiment:
at a constant pH of 1.4, the controlled release phase following the latent phase is such that the release time of 50% by weight of perindopril (t_{1/2}) is defined as follows (in hours): 0.25≦t_{1/2}≦35, preferably 0.5≦t_{1/2}≦20.

In practice, the release phase of the in vitro release profile of perindopril at a constant pH of 1.4 has a half-release time which is adjustable.

According to another interesting feature of the preferred embodiment:
the release phase following the transition from pH 1.4 to pH 6.8 is such that the release time of 50% by weight of perindopril (t_{1/2}) is defined as follows (in hours): 0.25≦t_{1/2}≦20, preferably 0.5≦t_{1/2}≦15.

Preferably, the microcapsules according to the invention comprise a single AB composite coating film. This simplifies their preparation and limits the amount of coating.

In the microcapsules according to the invention, perindopril will preferably be in the form of the tert-butylamine salt or arginine salt.

Preferably, perindopril is deposited on a neutral core having a diameter of from 50 to 600 microns.

Without there being any limitation, it has appeared desirable for the neutral core to be made of sucrose, dextrose, lactose or cellulose.

Advantageously, perindopril is deposited by techniques known to the person skilled in the art, for example the technique of spray coating in a fluidised air bed onto neutral cores of dextrose or sucrose having a diameter of from 200 to 600 microns.

With regard to quantities, the coating monolayer represents a maximum of 40% by weight, preferably a maximum of 30% by weight, of the microcapsules. Such a limited amount of film-coating allows galenic units to be obtained that each contain a high dose of soluble active ingredient without exceeding a size that would preclude its being swallowed. Observance, and thus success of the treatment, can only be found to be improved as a result.

The microcapsules described above may be used for the manufacture of new perindopril-based pharmaceutical compositions having optimised therapeutic performances which are preferably presented in the form of tablets (which are advantageously disintegrable and more especially even orodispersible), in the form of powders or in the form of gelatin capsules, preferably in the form of gelatin capsules.

Those microcapsules are all the more interesting because they are, in addition, perfectly tolerated by the organism, especially gastrically, and moreover can be obtained in an easy and economic manner.

The present invention relates furthermore to those new pharmaceutical compositions, in so far as they are novel in their structure, their presentation and their composition. The pharmaceutical compositions will preferably be administered by the oral route in the evening before going to bed.

It is to be noted that it may be of interest to mix, in the same gelatin capsule, same tablet or same powder, at least two types of microcapsules that have different release kinetics but are included within the characteristic framework of the invention.

It is likewise possible to mix the microcapsules according to the invention with a certain amount of perindopril that is immediately available in the organism.

A combination of the microcapsules containing perindopril and microcapsules containing active ingredients other than perindopril is also possible. By way of preference, it will be possible for indapamide microcapsules to be combined with perindopril microcapsules.

Those pharmaceutical compositions, obtained starting from microcapsules according to the invention, are beneficial for the treatment of arterial hypertension and heart failure.

A clinical study carried out with patients using gelatin capsules containing the microcapsules according to the invention administered at about 2200 hours has shown that the plasma concentrations of active ingredient were such that they enabled a substantial reduction in the pressure rise observed in the mornings and enabled the pressure control over that period to be improved.

On the other hand, it was demonstrated in the clinical study that, using the microcapsules according to the invention, pressure protection was perfect over the whole of the nychthemeron, that the number of patients having normalised arterial pressure was higher than that obtained with an immediate-release tablet and, finally, that there was a net improvement in interindividual variation.

Finally, it was observed in the clinical study that, unlike the immediate-release tablet currently on the market, which must be taken before meals, the intake of food changing the bioavailability of the active ingredient, the pharmaceutical compositions according to the invention can be administered before or after meals without the bioavailability being changed.

The examples of formulations of perindopril microcapsules below illustrate the invention but do not limit it in any way.

1—Preparation of Perindopril Microcapsules

Step A: Preparation of Perindopril Microparticles

157 g of perindopril tert-butylamine salt and 17 g of hydroxypropyl cellulose are dispersed or dissolved in 1300 g of acetone. The suspension is sprayed in a Glatt GPCG3 spray coater onto 1500 g of sugar microspheres having an average diameter of from 355 to 500 μm. The film-coating conditions are: product temperature: 37-39° C., spray delivery rate: 42 g/min, atomisation pressure: 1.8 bar.

Step B: Preparation of Perindopril Microcapsules

The hydrophilic polymer A and the hydrophobic compound B are dissolved in isopropanol heated to a temperature of from 65 to 75° C. The solution is sprayed in a Glatt GPCG3 spray coater onto the perindopril microparticles prepared in Step A. The film-coating conditions are: product temperature: 36-41° C., spray delivery rate: 8-12 g/min, atomisation pressure: 1.5 bar.

2- Formulation Examples
The formulation examples are given in the Table below:

| | Microcapsule constituents | | |
|---|---|---|---|
| | Perindopril | Polymer A | Compound B |
| | microparticles | nature/quantity | nature/quantity |
| | (g) | (g) | (g) |
| Formulation | 700 | Eudragit ® L100 | Hydrogenated palm oil |
| 1 | | 37 | 56 |
| Formulation | 700 | Eudragit ® L100 | Hydrogenated palm oil |
| 2 | | 140 | 93 |
| Formulation | 700 | Eudragit ® L100 | Hydrogenated palm oil |
| 3 | | 93 | 140 |
| Formulation | 700 | Eudragit ® L100 | Hydrogenated palm oil |
| 4 | | 105 | 70 |
| Formulation | 700 | Eudragit ® | Hydrogenated |
| 5 | | L100-55 | cottonseed oil |
| | | 105 | 70 |

Remarks: the quantities of isopropanol used for the preparation of formulations 1 to 5 are, respectively, 840 g, 2100 g, 2100 g, 1576 g and 1575 g.

The microcapsules of formulations 1 to 5 were tested in a Dissolutest, according to the pharmacopoeia, maintained at 37° C. and stirred at 100 revs/min at constant pH or with evolving pH.

The results of those tests are presented in the accompanying FIGS. 1 to 5.

FIG. 1: Formulations 1 and 2

These microcapsules were tested in HCl medium at pH 1.4. The release profiles obtained with the two formulations are characteristic of a delayed and prolonged release. For both formulations, the release phase is triggered without change of pH after the respective latent periods of 1 and 3 hours. It will be noted that, despite the different latent periods, appropriate choices of the Eudragit® L100/palm oil ratios allowed similar release kinetics to be obtained in the release phase.

FIG. 2: Formulation 2

These microcapsules were tested in an HCl medium at pH 1.4 for 3 hours and at pH 6.8 subsequently.

The release profiles are characteristic of a delayed and prolonged release. The release phase is triggered on change of the pH at t=3 hours. Comparative examination of the release profiles at pH 1.4 and with evolving pH thus demonstrate that the release can be triggered by a change of pH or without a change of pH.

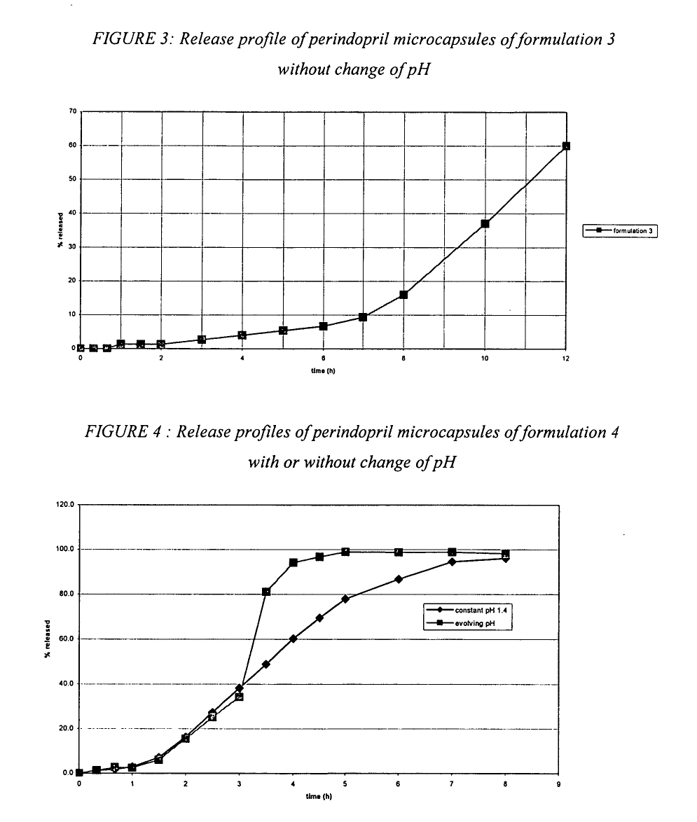

FIG. 3: Formulation 3

These microcapsules were tested in an HCl medium at pH 1.4. The release profile is characteristic of a delayed and prolonged release. The release phase is triggered without change of pH after a latent period of 6 hours

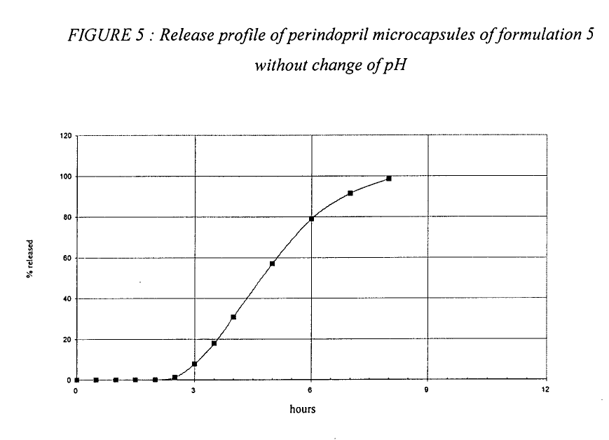

FIG. 4: Formulation 4

These microcapsules were tested either at constant pH (1.4) or with evolving pH (1.4 for 3 hours then 6.8). The release profiles are characteristic of a delayed and prolonged release. It is confirmed that, with evolving pH, the release phase is triggered twice, at t=1 without change of pH, then the increase in pH at t=3 hours triggers the second release mechanism after the pH has changed.

FIG. 5: Formulation 5

These microcapsules were tested at pH 1.4. The release profile is characteristic of a delayed and prolonged release. The release phase is triggered without change of pH after a latent period of 2.5 hours.

1-16. (canceled)

17- A “Reservoir” microcapsule composition for the delayed and controlled release of perindopril or a pharmaceutically acceptable salt thereof for oral administration, comprising:
microparticles of perindopril or a pharmaceutically acceptable salt thereof, wherein the microparticles are each covered by at least one coating film, the coating film being formed from a composite material comprising:
at least one hydrophilic polymer A carrying groups ionised at neutral pH,
at least one hydrophobic compound B, representing a mass fraction (% by weight in relation to the total mass of the microcapsule) less than or equal to 40,
wherein the microparticles have a diameter of less than 1200 microns.

18- A microcapsule composition according to claim 17, wherein the hydrophilic polymer A is selected from cellulose compounds, copolymers of methacrylic acid and a methacrylic acid ester, copolymers of methacrylic acid and an acrylic acid ester and mixtures thereof.

19- A microcapsule compostion according to claim 18, wherein the hydrophilic polymer A is a copolymer of methacrylic acid and methyl methacrylate or a copolymer of methacrylic acid and ethyl acrylate.

20- A microcapsule composition according to claim 17, wherein the hydrophobic compound B is selected from vegetable waxes, hydrogenated vegetable oils, hydrogenated triglycerides and mixtures thereof.

21- A microcapsule composition according to claim 17, wherein the hydrophobic compound B is a hydrogenated vegetable oil.

22- A microcapsule composition according to claim 17, wherein the coating film is composed of a mixture of hydrophilic polymer A and hydrophobic compound B in which the weight ratio B/A is between 0.2 and 4.

23- A microcapsule composition according to claim 17, wherein the coating film enables:
at a pH of 1.4, a dissolution profile comprising a latent phase of a duration greater than or equal to half an hour, to be obtained,
a release phase of perindopril to be obtained at any instant during the latent phase after transition from pH 1.4 to pH 6.8.

24- A microcapsule composition according to claim 23, wherein the latent phase is from 1 to 8 hours.

25- A microcapsule composition according to claim 23, wherein the latent phase is from 1 to 5 hours.

26- A microcapsule composition according to claim 17, wherein perindopril is in the form of a tert-butylamine salt.

27- A microcapsule composition according to claim 17, wherein perindopril is in the form of a arginine salt.

28- A microcapsule composition according to claim 26, wherein perindopril or a pharmaceutically acceptable salt thereof is deposited onto a neutral core having a diameter of from 50 to 600 microns.

29- A microcapsule composition according to claim 27, wherein perindopril or a pharmaceutically acceptable salt thereof is deposited onto a neutral core having a diameter of from 50 to 600 microns.

30- A microcapsule composition according to claim 28, wherein the neutral hydrophilic core is made of sucrose, dextrose, lactose or cellulose.

31- A microcapsule composition according to claim 29, wherein the neutral hydrophilic core is made of sucrose, dextrose, lactose or cellulose.

32- A microcapsule composition according to claim 17, wherein the perindopril microcapsules are combined with indapamide microcapsules.

33- A method of administration of an active principle to a patient in need thereof, comprising administration of the active principle in a microencapsulated form as a component of a tablet, powder, or gelatine capsule.

34- A pharmaceutical composition comprising as active principle an effective amount of a microcapsule composition according to claim 17 together with one or more pharmaceutically-acceptable excipients or vehicles.

35- A pharmaceutical composition according to claim 33 wherein the pharmaceutical composition is in the form of a tablet, powder, or gelatine capsule.

36- A pharmaceutical composition according to claim 34 wherein the pharmaceutical composition is in the form of a gelatin capsule.

37- A method for treating a living animal body, including a human, afflicted with a condition selected from arterial hypertension and heart failure, comprising the step of administering to the living animal body, including a human, an amount of a pharmaceutical composition according to claim 33 which is effective for alleviation of the condition.
